(19) United States
(12) Patent Application Publication
(10) Pub. No.: US 2026/0041889 A1
(43) Pub. Date: Feb. 12, 2026

(54) CATHETER DRAINAGE BAG OR WOUND CARE DRAINAGE BAG SUPPORTING APPARATUS, AND A WHEELCHAIR WITH A CATHETER DRAINAGE BAG OR WOUND CARE DRAINAGE SUPPORTING APPARATUS

(71) Applicants: Gilbert James McLaughlin, Tucson, AZ (US); David Michael Kriesel, Rancho Santa Fe, CA (US)

(72) Inventors: Gilbert James McLaughlin, Tucson, AZ (US); David Michael Kriesel, Rancho Santa Fe, CA (US)

(21) Appl. No.: 18/795,765

(22) Filed: Aug. 6, 2024

(51) Int. Cl. A61M 25/02; A61G 5/10; A61M 27/00

(52) U.S. Cl. CPC A61M 25/02; A61G 5/10; A61M 27/00; A61M 2209/082

(57) ABSTRACT

An apparatus for supporting a catheter drainage bag or wound care drainage bag on a wheelchair includes an upper arm having a free end and a second end, a lower arm having an end connected to the second end of the upper arm and a supporting end, a cross bar extending perpendicular to longitudinal dimensions of the upper arm and the lower arm, and suspending arms extending between the supporting end of the lower arm and ends of the cross bar. A wheelchair with a catheter drainage bag or wound care drainage bag supporting apparatus is also provided.

### BACKGROUND AND SUMMARY

The present invention relates generally to supporting apparatus and, more particularly, to apparatus for supporting a catheter drainage bag or a wound care drainage bag relative to a wheelchair.

Drainage bags are typically connected to catheters such as Foley catheters and wound care systems by one or more tubes. The drainage bag and the tubes can be inconvenient, particular when a user must also use a wheelchair. There is ordinarily no convenient place to put the drainage bag, and it is not uncommon for the drainage bag or tubes to become tangled in the wheelchair components or the user's limbs which can be dangerous and painful. It is known to suspend drainage bags in pouches or bags suspended on the wheelchair, typically below the seat of the wheelchair. These pouches or bags can conceal the drainage bags and locate them out of the way, but are not typically conveniently located and often require complex straps and buckles to attach to a wheelchair.

In accordance with an aspect of the present invention, an apparatus for supporting a catheter drainage bag or wound care drainage bag on a wheelchair comprises an upper arm having a free end and a second end, a lower arm having an end connected to the second end of the upper arm and a supporting end, a cross bar extending perpendicular to longitudinal dimensions of the upper arm and the lower arm, and suspending arms extending between the supporting end of the lower arm and ends of the cross bar.

In accordance with another aspect of the present invention, a wheelchair with a catheter drainage bag or wound care drainage bag supporting apparatus comprises a wheelchair including a seat portion, and the catheter drainage bag or wound care drainage bag supporting apparatus, comprising an upper arm having a free end and a second end, a lower arm having an end connected to the second end of the upper arm and a supporting end, a cross bar extending perpendicular to longitudinal dimensions of the upper arm and the lower arm, and suspending arms extending between the supporting end of the lower arm and ends of the cross bar, wherein the upper arm and the lower arm are disposed on opposite sides of the seat portion so that the cross bar is disposed rearwardly of a front end of the seat portion.

### BRIEF DESCRIPTION OF THE DRAWINGS

The features and advantages of the present invention are well understood by reading the following detailed description in conjunction with the drawings in which like numerals indicate similar elements and in which:

FIG. 1A is a side view of a portion of a wheelchair seat to which a bag supporting apparatus is attached according to an aspect of the present invention;

FIG. 1B is a side view of a portion of a wheelchair seat to which a bag supporting apparatus is attached according to another aspect of the present invention;

FIG. 2 is a perspective view of a bag supporting apparatus according to an aspect of the present invention;

FIG. 3 is a side view of the bag supporting apparatus of FIG. 2;

FIG. 4 is a top view of the bag supporting apparatus of FIG. 2; and

FIG. 5 is a front view of the bag supporting apparatus of FIG. 2.

### DETAILED DESCRIPTION

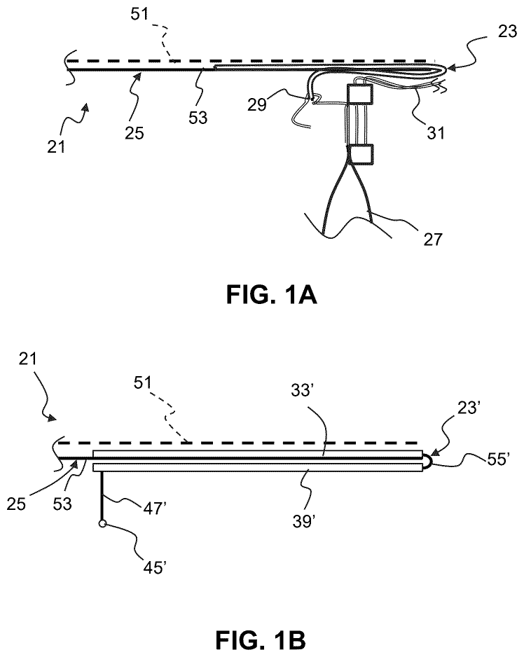

A relevant portion of a wheelchair 21 with a catheter drainage bag or wound care drainage bag supporting apparatus 23 is shown in FIG. 1A. The wheelchair 21 may be a wheelchair of any suitable well-known design including a seat portion 25. For example, in addition to the seat portion 25, which will ordinarily be a flexible fabric, plastic, or similar material secured between a pair of parallel, horizontal bars of a frame of the wheelchair, the wheelchair will typically include front rigging attached to a forward portion of the frame with a footrest and leg rest attached to the front rigging, caster wheels attached to the frame outside of the front rigging, large rear wheels attached near a rear of the frame, a seat back or cane portion comprising a pair of parallel, substantially vertical bars of the frame with a flexible fabric, plastic, or similar material secured between the vertical bars, and push handles at top ends of the vertical bars. The frame will typically also include armrests and, beneath the seat, cross bars extending diagonally between upper and lower pairs of horizontal bars and that provide rigidity to the frame. The rear wheels will typically also include hand rims.

A relevant portion of a catheter drainage bag or wound care drainage bag 27 is also shown in FIG. 1A. Such a bag 27 will ordinarily be of a type comprising some form of clip 29 that is adapted to be clipped around a bar or rail. One or more drainage lines 31 will typically be connected to a catheter such as a Foley catheter or fistula management system (not shown).

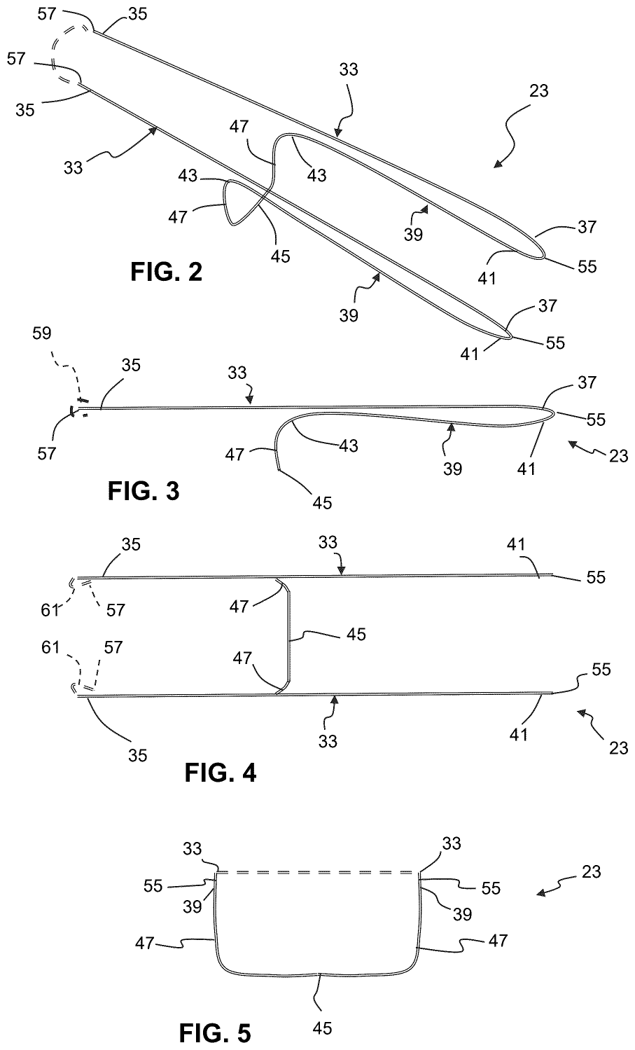

The catheter drainage bag or wound care drainage bag supporting apparatus 23 is shown in more detail in FIGS. 2-5. As seen, for example, in FIG. 2, the bag supporting apparatus 23 comprises an upper arm 33 having a free end 35 and a second end 37. The bag supporting apparatus 23 further comprises a lower arm 39 having an end 41 connected to the second end 37 of the upper arm 33 and a supporting end 43. A cross bar 45 extends perpendicularly to longitudinal dimensions of the upper arm 33 and the lower arm 39. Suspending arms 47 extend between the supporting end 43 of the lower arm 39 and ends 49 of the cross bar 45.

As seen in FIG. 1, the upper arm 33 and the lower arm 39 are disposed on opposite sides of the seat portion 25 so that the cross bar 45 is disposed rearwardly of a front end 49 of the seat portion 25. The clip 29 on the bag 27 will be attached around the cross bar 45 so that the bag will be disposed beneath the seat 25 and remote from the front end 49 of the seat so that the likelihood of unintentional disturbance of the bag is minimized.

In certain wheelchairs 21, the seat portion 25 includes an upper panel 51 (shown in phantom in FIG. 1A) and a lower panel 53. The upper arm 33 of the bag supporting apparatus 23 can be fit between the upper panel 51 and the lower panel 53 of the seat portion 25, concealing part of the bag supporting apparatus 23 and reducing the possibility of discomfort for the user.

The upper arm 33 and the lower arm 39 of the bag supporting apparatus 23 are ordinarily connected to each other by a connecting member 55 that maintains a spatial relationship between the upper arm and the lower arm. The spatial relationship is ordinarily sufficient to permit the part of the seat portion 25 to which the bag supporting apparatus 23 is attached to be easily installed on and removed from the seat portion, and the upper arm 33 and the lower arm 39 are ordinarily sufficiently long to prevent the bag supporting apparatus from accidentally sliding off the seat portion, particularly when held in place by the weight of a person seated in the wheelchair. The upper arm 33 and the lower arm 39 will ordinarily be spaced sufficiently to permit easy manual movement of bag supporting apparatus 23 relative to the part of the seat portion between them, but they may, if desired or necessary, be sufficiently close to pinch the part of the seat portion between them so that there is some degree of resistance to movement of the bag supporting apparatus.

As seen in FIGS. 2-5, in a presently preferred embodiment, the upper arm 33, the connecting member 55, the lower arm 39, the cross bar 45, and the suspending arms 47 are formed from bent wire, preferably a single, continuous piece of bent wire. As seen in FIGS. 3 and 4, the free end 35 of the upper arm can comprise opposite ends 57 of the piece of wire. If the opposite ends 57 of the piece of wire are disposed at the free end 35 of the upper arm 33, covers 59 (shown in phantom in FIG. 3) may be provided on the opposite ends of the piece of wire, or tips 61 (shown in phantom in FIG. 4) of the opposite ends of the piece of wire may be bent. In this way, any pointed or sharp ends of the wire can be blunted, reducing the chance of discomfort for the user. The wire can have two ends as shown in FIGS. 2-5 or the bent wire can be an endless wire as shown in phantom in FIGS. 2 and 5. The endless wire can be formed with no ends, or opposite ends of a wire can be joined together, such as by being intertwined. An endless wire can minimize the chance of discomfort for the user and may strengthen the bag supporting apparatus 23.

The connecting member 55 can be a part of a continuous wire as shown in FIGS. 2-5. The connecting member 55 may also be a spring member. If the connecting member 55 is part of a continuous wire, the connecting member will ordinarily transition to the upper arm 33 and the lower arm 39 without a distinct boundary between the parts of the bag supporting apparatus. The connecting member 55 will ordinarily be sufficiently resilient to permit the upper arm 33 and the lower arm 39 to be separated to at least a sufficient degree to receive the seat portion 25 between them and then return to substantially their original position.

Instead of forming the bag supporting apparatus 23 from a single, continuous piece of bent wire, at least one of the upper arm 33′ and the lower arm 39′ can be a plate as shown in FIG. 1B. The connecting member 55′ between the upper arm 33′ and the lower arm 39′ can be a wire, or a plate, or any desired structure that can hold the upper arm and the lower arm relative to each other, while permitting the upper arm and the lower arm to separate sufficiently from each other to permit a seat portion 25 between them.

In the present application, the use of terms such as “including” is open-ended and is intended to have the same meaning as terms such as “comprising” and not preclude the presence of other structure, material, or acts. Similarly, though the use of terms such as “can” or “may” is intended to be open-ended and to reflect that structure, material, or acts are not necessary, the failure to use such terms is not intended to reflect that structure, material, or acts are essential. To the extent that structure, material, or acts are presently considered to be essential, they are identified as such.

While this invention has been illustrated and described in accordance with a preferred embodiment, it is recognized that variations and changes may be made therein without departing from the invention as set forth in the claims.

What is claimed is:

1. An apparatus for supporting a catheter drainage bag or wound care drainage bag on a wheelchair, comprising:
an upper arm having a free end and a second end;
a lower arm having an end connected to the second end of the upper arm and a supporting end;
a cross bar extending perpendicular to longitudinal dimensions of the upper arm and the lower arm; and
suspending arms extending between the supporting end of the lower arm and ends of the cross bar.

2. The apparatus as set forth in claim 1, wherein the upper arm and the lower arm are connected to each other by a connecting member that maintains a spatial relationship between the upper arm and the lower arm.

3. The apparatus as set forth in claim 2, wherein the upper arm, the connecting member, the lower arm, the cross bar, and the suspending arms are formed from bent wire.

4. The apparatus as set forth in claim 3, wherein the bent wire is a continuous piece of wire.

5. The apparatus as set forth in claim 4, wherein the free end of the upper arm comprises opposite ends of the piece of wire.

6. The apparatus as set forth in claim 5, comprising covers on the opposite ends of the piece of wire.

7. The apparatus as set forth in claim 4, wherein tips of the opposite ends of the piece of wire are bent.

8. The apparatus as set forth in claim 4, wherein the bent wire is an endless wire.

9. The apparatus as set forth in claim 1, wherein the connecting member is a spring member.

10. The apparatus as set forth in claim 9, wherein at least one of the upper arm and the lower arm is a plate.

11. The apparatus as set forth in claim 1, wherein the upper arm, the lower arm, the cross bar, and the suspending arms are formed from bent wire.

12. The apparatus as set forth in claim 11, wherein the bent wire is a continuous piece of wire.

13. The apparatus as set forth in claim 11, wherein the free end of the upper arm comprises opposite ends of the piece of wire.

14. The apparatus as set forth in claim 12, comprising covers on the opposite ends of the piece of wire.

15. The apparatus as set forth in claim 14, wherein tips of the opposite ends of the piece of wire are bent.

16. The apparatus as set forth in claim 11, wherein the bent wire is an endless wire.

17. A wheelchair with a catheter drainage bag or wound care drainage bag supporting apparatus, comprising:
a wheelchair including a seat portion;
the catheter drainage bag or wound care drainage bag supporting apparatus, comprising
an upper arm having a free end and a second end,
a lower arm having an end connected to the second end of the upper arm and a supporting end,
a cross bar extending perpendicular to longitudinal dimensions of the upper arm and the lower arm, and
suspending arms extending between the supporting end of the lower arm and ends of the cross bar,
wherein the upper arm and the lower arm are disposed on opposite sides of the seat portion so that the cross bar is disposed rearwardly of a front end of the seat portion.

18. The wheelchair with the catheter drainage bag or wound care drainage bag supporting apparatus as set forth in claim 17, wherein the upper arm and the lower arm are connected to each other by a connecting member that maintains a spatial relationship between the upper arm and the lower arm.

19. The wheelchair with the catheter drainage bag or wound care drainage bag supporting apparatus as set forth in claim 17, wherein the seat portion includes an upper panel and a lower panel, the upper arm of the catheter drainage bag supporting apparatus being disposed between the upper panel and the lower panel of the seat portion.

20. The wheelchair with the catheter drainage bag or wound care drainage bag supporting apparatus as set forth in claim 19, wherein the upper arm and the lower arm are connected to each other by a connecting member that maintains a spatial relationship between the upper arm and the lower arm.
